(19) United States
(12) Patent Application Publication
(10) Pub. No.: US 2005/0239756 A1
(43) Pub. Date: Oct. 27, 2005

(54) Methods of treating various vitamin D metabolism conditions with 1alpha-hydroxyvitamin D2

(75) Inventors: Charles W. Bishop, Mount Horeb, WI (US); Eric Messner, Verona, WI (US); Keith Crawford, Fitchburg, WI (US)

Correspondence Address: MICHAEL BEST & FRIEDRICH, LLP, ONE SOUTH PINCKNEY STREET, P O BOX 1806, MADISON, WI, 53701, US

(73) Assignee: Bone Care International, Inc., Middleton, WI (US)

(21) Appl. No.: 11/113,738

(22) Filed: Apr. 25, 2005

Related U.S. Application Data
(60) Provisional application No. 60/564,915, filed on Apr. 23, 2004.

(51) Int. Cl. A61K 31/59

(52) U.S. Cl. 514/167

(57) ABSTRACT

The present invention provides a method of increasing or maintaining blood concentrations of 1,25-dihydroxyvitamin D in a patient by administering an amount of 1α-hydroxyvitamin D₂. The invention also provides a method of concurrently lowering or maintaining plasma intact parathyroid hormone levels, increasing or maintaining serum calcium levels, maintaining serum phosphorous levels, or increasing or maintaining serum 1,25-dihydroxyvitamin D levels in a human patient by administering to the patient an amount of 1α-hydroxyvitamin D₂. The invention also provides a method of reducing the risk of over suppression of plasma intact parathyroid hormone levels in a patient undergoing treatment for elevated levels of plasma intact parathyroid hormone, by administering 1α-hydroxyvitamin D₂ in an amount sufficient to decrease elevated intact parathyroid hormone levels while avoiding an abnormally low bone turnover rate.

### CROSS REFERENCE TO RELATED APPLICATIONS

This application claims priority to U.S. provisional application No. 60/564,915 filed Apr. 23, 2004. This application is incorporated by reference in it's entirety.

### BACKGROUND

“Vitamin D” is a general term for ergocalciferol (Vitamin D₂), cholecalciferol (Vitamin D₃) and related metabolites that are essential for Ca, P and parathyroidhormone (PTH) homeostasis in healthy individuals. Hepatic enzymes convert ergocalciferol and cholecalciferol to their 25-hydroxylated forms which undergo a second hydroxylation catalyzed by the renal 1α-hydroxylase to form the two major vitamin D hormones known as calcitriol (1,25-dihydroxyvitamin D₃) and 1,25-dihydroxyvitamin D₂. Together, these two hormones are referred to as “1,25-dihydroxyvitamin D”.

The renal 1α-hydroxylase which is used to form 1,25-dihydroxyvitamin D is stimulated by parathyroid hormone (PTH) which is secreted by the parathyroid glands, under tight feedback regulation, to increase production of 1,25-dihydroxyvitamin D. In young normal adults, blood levels of 1,25-dihydroxyvitamin D remain stable, varying up or down by a few pg/mL throughout each day (and even each year) generally within the range of 20 to 60 pg/mL. Some individuals have normal levels of 1,25-dihydroxyvitamin D that stay near the upper end of this range while others have normal levels that hover closer to the lower end. Due to the great variation in normal values of healthy adults, it is important that each individual's serum 1,25-dihydroxyvitamin D levels be checked periodically (typically annually or biannually). when the kidneys are healthy, so that changes in serum 1,25-dihydroxyvitamin D levels can be more accurately evaluated.

Various disorders in vitamin D metabolism and related abnormalities in mineral and bone metabolism can lead to deficiencies of 1,25-dihydroxyvitamin D. Chronic 1,25-dihydroxyvitamin D deficiency elicits prolonged and steadily increasing secretion of intact PTH. It also fosters inadequate intestinal Ca absorption, causing reduced extracellular Ca concentrations, which further stimulate PTH secretion. Eventually, the overstimulated parathyroid glands become hyperplastic and develop increasing resistance to regulation by vitamin D hormone therapies.

Deficiencies in 1,25-dihydroxyvitamin D in a human can be caused by environmental conditions where sunlight exposure is limited. Because vitamin D is a fat-soluble vitamin, conditions that reduce digestion or absorption of fats decrease the ability of vitamin D to be absorbed from the intestines and subsequently cannot be used to produce 1,25-dihydroxyvitamin D.

Another disorder that leads to deficiencies of 1,25-dihydroxyvitamin D is chronic kidney disease (CKD). CKD includes conditions that affect the kidney, with the potential to cause either progressive loss of kidney function or complications resulting from decreased kidney function. CKD is defined as either kidney damage or glomerular filtration rate (GFR) of less than 60 mL/min/1.73 m² for more than three months. CKD is now classified in stages based on the level of kidney function, as defined by estimated GFR. Stage 1 is defined as normal kidney function with some kidney damage and a GFR of ≧90 mL/min/1.73 m²; Stage 2 involves mildly decreased kidney function with a mild decrease in GFR, i.e., a GFR of 60-89 mL/min/1.73 m . Stage 3 is defined as moderately decreased kidney function with a GFR of 30-59 mL/min/1.73 m². Stage 4 is defined as severely decreased kidney function with a GFR of 15-29 mL/min/1.73 m². Stage 5 is kidney failure with a GFR of <15-29 mL/min/1.73 m² or dialysis (end-stage renal disease or ESRD).

The activity of the renal 1α-hydroxylase progressively declines in CKD. It falls predictably with the steady loss of functioning nephrons as GFR falls below approximately 60 ml/min/1.73 m², leading to a relative and, subsequently, an absolute deficiency of serum 1,25-dihydroxyvitamin D. The activity of any remaining renal 1α-hydroxylase is directly inhibited by rising serum phosphorous caused by progressively inadequate renal elimination of dietary phosphorous.

The consequences of chronic 1,25-dihydroxyvitamin D deficiency are far reaching and have recently been linked to increased mortality in ESRD patients. Osteitis fibrosa cystica, or rapid turnover bone disease characterized by increased osteoblastic and osteoclastic activity, has been the most commonly observed form of bone disease in patients with CKD. More recently, adynamic bone disease, which is associated with reduced bone turnover and low levels of PTH, has increased in prevalence, particularly among patients on dialysis. Increased dietary intake of Ca, high doses of aluminum-based phosphate binders, and parathyroidectomy have contributed to this rise. Ironically, overuse of vitamin D hormone therapies has also contributed to the more frequent development of adynamic bone disease.

The present invention provides novel therapies for treating elevated levels of parathyroid hormone and decreased levels of 1,25-dihydroxyvitamin D₂.

### BRIEF DESCRIPTION OF THE INVENTION

In one aspect the present invention provides a method of increasing or maintaining blood concentrations of 1,25-dihydroxyvitamin D in a patient by administering an amount of 1α-hydroxyvitamin D₂. The blood concentrations of 1,25-dihydroxyvitamin D are increased to or maintained within a patient's normal historical physiological range for 1,25-dihydroxyvitamin D without causing substantially increased risk of hypercalcemia, hyperphosphatemia or over suppression of plasma intact parathyroid hormone in the patient. The blood levels of 1,25-dihydroxyvitamin D are maintained in the patient's normal historical physiological range between doses of 1α-hydroxyvitamin D₂.

In another aspect, the invention provides a method of concurrently lowering or maintaining plasma intact parathyroid hormone levels, increasing or maintaining serum calcium levels, maintaining serum phosphorous levels, or increasing or maintaining serum 1,25-dihydroxyvitamin D levels in a human patient by administering to the patient an amount of 1α-hydroxyvitamin D₂.

In yet another aspect, the invention provides a method of reducing the risk of over suppression of plasma intact parathyroid hormone levels in a patient undergoing treatment for elevated levels of plasma intact parathyroid hormone, by administering 1α-hydroxyvitamin D₂ in an amount sufficient to decrease elevated intact parathyroid hormone levels while avoiding an abnormally low bone turnover rate.

A fuller appreciation of the specific attributes of this invention will be gained upon an examination of the following detailed description of preferred embodiments, and appended claims.

### BRIEF DESCRIPTION OF THE DRAWINGS

FIG. 1 is a chart showing changes of plasma intact PTH (iPTH) levels, shown as percent of Baseline value, in subjects given doxercalciferol (1αD₂) compared to those given placebo in Example 2.

FIG. 2 is a chart showing corrected serum calcium (S-Ca) and serum phosphorus (S-P) in subjects receiving doxercalciferol (1αD₂) compared to those receiving placebo in Example 2.

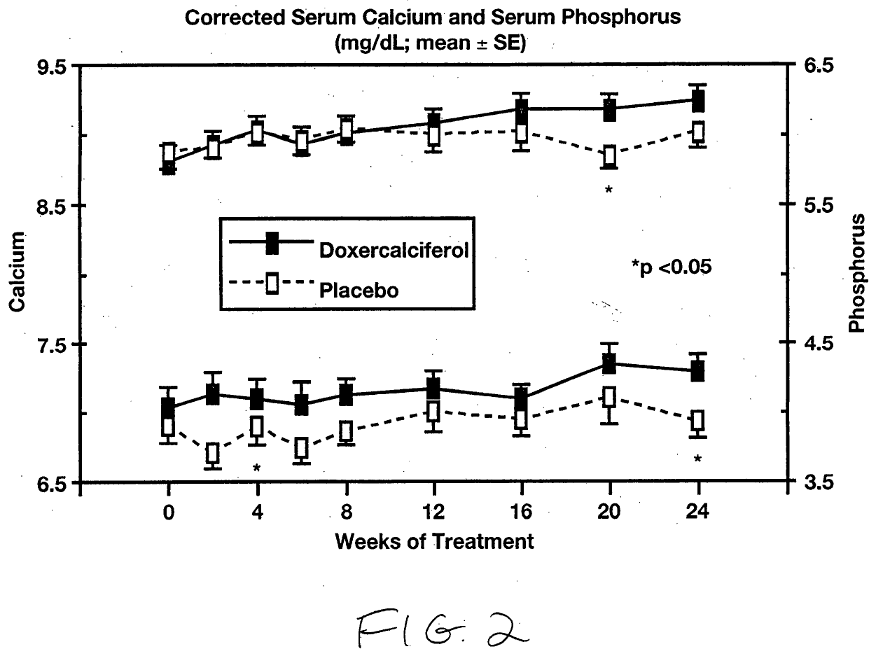

FIG. 3 is a chart showing fasting spot urine calcium/creatinine ratios (Left) and 24 hour urine calcium (Right) in the group assigned to doxercalciferol (1αD₂) compared to that assigned to placebo in Example 2.

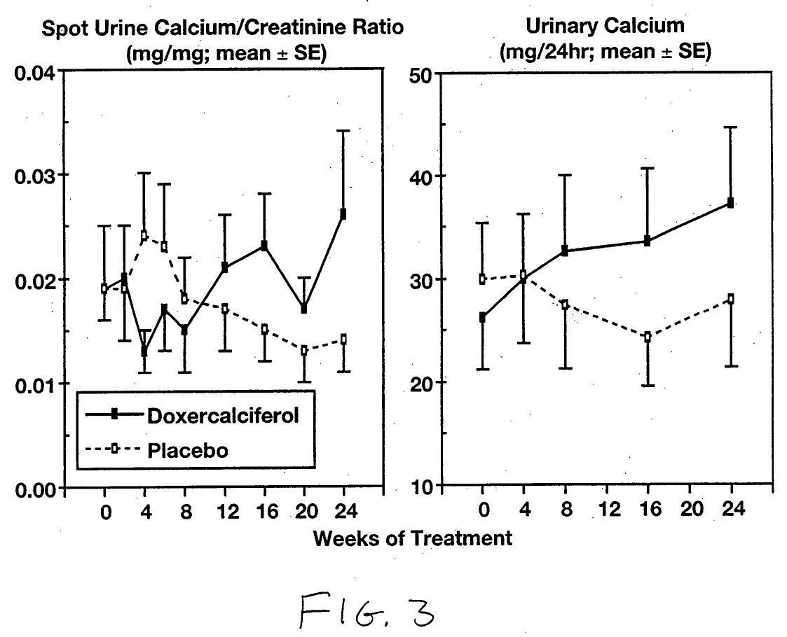

FIG. 4 is a chart showing glomerular filtration rates determined by either technetium-labeled diethylene triamine pentacetic acid (DTPA) or I¹²⁵-labeled Iothalamate at Baseline and at the last study visit (Week 24) in 22 doxercalciferol-treated subjects (Left) compared to 20 placebo-treated subjects (Right). The Baseline and Week 24 mean values (±SD) are shown as vertical bars in Example 2.

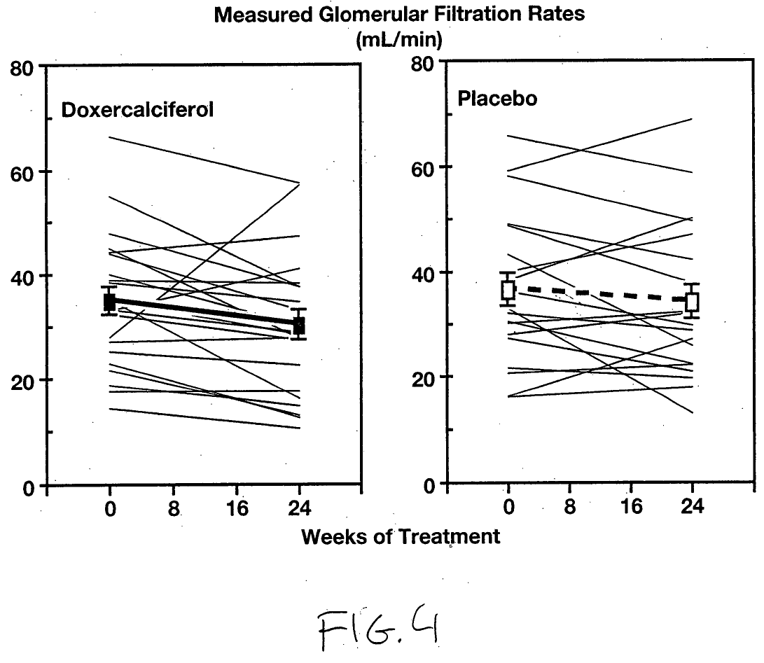

FIG. 5 is a chart showing changes in serum total 1,25-dihydroxyvitamin D from Baseline in subjects receiving doxercalciferol (1αOH D₂) compared to those receiving placebo.

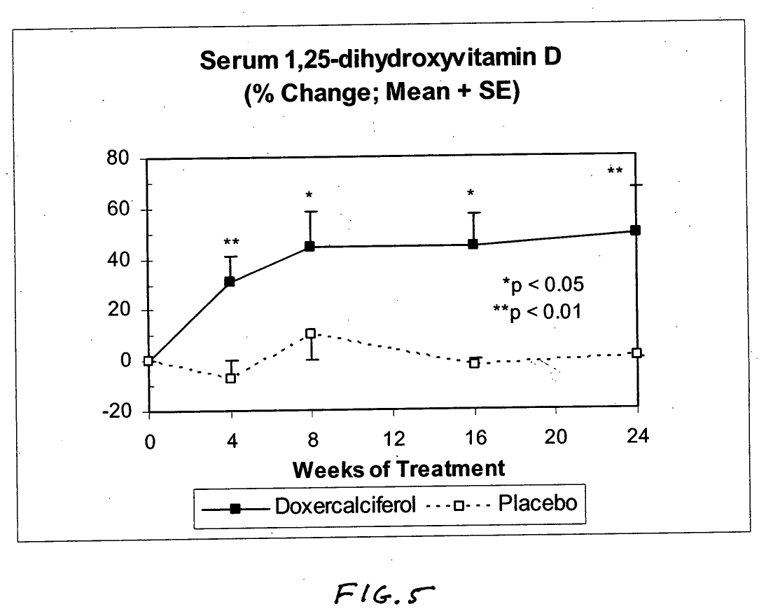

FIG. 6 is a chart showing the biochemical profile (mean±SD) of enrolled subjects at baseline by treatment group in Example 2.

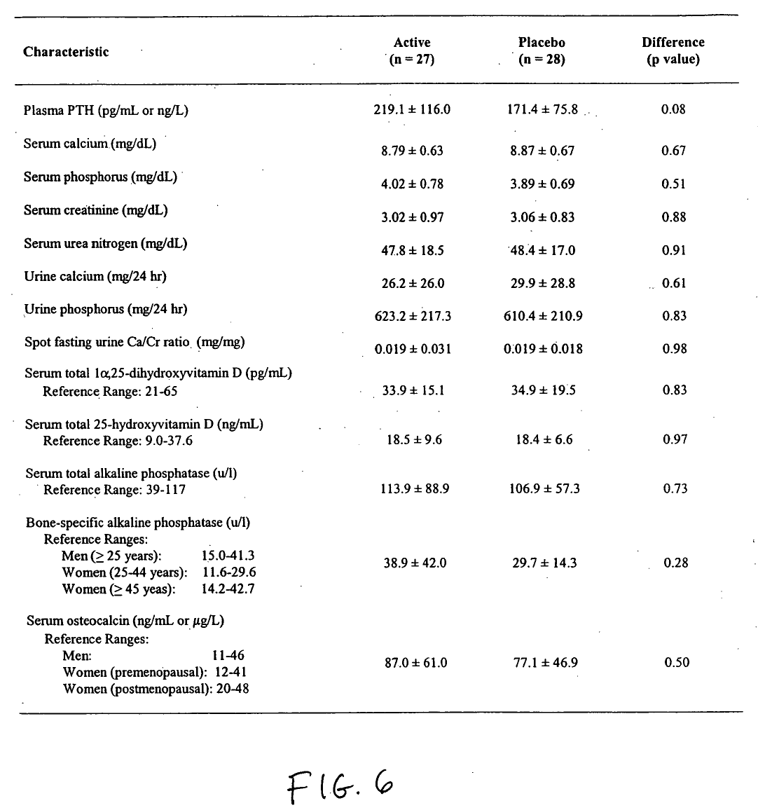

FIG. 7 is a chart showing the comparison of elevated values of corrected serum calcium, serum phosphorus, 24-hour urine calcium and fasting spot urine calcium:creatinine ratio during treatment with doxercalciferol and placebo in Example 2.

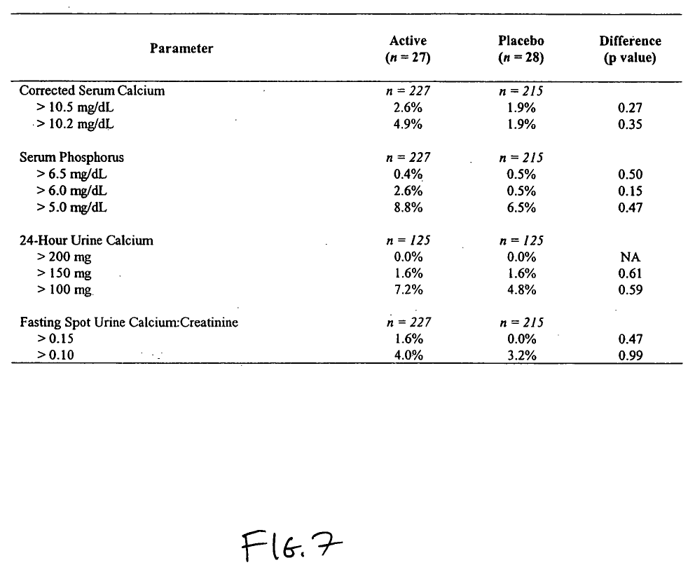

Before the embodiments of the invention are explained in detail, it is to be understood that the invention is not limited in its application to the details of construction and the arrangements of the components set forth in the following description or illustrated in the drawings. The invention is capable of other embodiments and of being practiced or being carried out in various ways. Also, it is understood that the phraseology and terminology used herein are for the purpose of description and should not be regarded as limiting. The use of “including”, “having” and “comprising” and variations thereof herein is meant to encompass the items listed thereafter and equivalents thereof as well as additional items and equivalents thereof.

### DETAILED DESCRIPTION OF THE INVENTION

The present invention relates to treating various disorders in vitamin D metabolism and related abnormalities in mineral and bone metabolism by administering an effective amount of 1α-hydroxyvitamin D₂.

In one aspect the present invention provides a method of increasing or maintaining blood concentrations of 1,25-dihydroxyvitamin D in a patient by administering an amount of 1α-hydroxyvitamin D₂. As noted hereinbefore, many conditions can lead to 1,25-dihydroxyvitamin D deficiencies, including living in northern latitudes. It has been found that treatment with 1α-hydroxyvitamin D₂ of those patients in need thereof can provide blood concentrations of 1,25-dihydroxyvitamin D that are increased or maintained within a patient's normal historical range for 1,25-dihydroxyvitamin D. such administration is accomplished without a substantially increased risk of hypercalcemia, hyperphosphatemia or over suppression of plasma intact pararthyroid hormone (PTH), all of which have been recognized as risks when treatment with a vitamin D compound is incurred. Moreover, blood levels of 1,25-dihydroxyvitamin D are maintained in the patients historical physiological range between doses, eliminating spike and trough concentration patterns.

In another aspect, the invention provides a method of concurrently lowering or maintaining plasma intact parathyroid hormone levels, increasing or maintaining serum calcium levels, maintaining serum phosphorous levels, or increasing or maintaining serum 1,25-dihydroxyvitamin D levels in a human patient by administering to the patient an amount of 1α-hydroxyvitamin D₂. Many diseases manifest abnormal levels of more than one hormone and mineral. In CKD, for example, patients may experience decreases in 1,25-dihydroxyvitamin D, increases in PTH and increases in serum phosphorous. Treatment in accordance with the present invention presents concurrent leveling and/or maintaining of these carious hormone and mineral levels.

In yet another aspect, the invention provides a method of reducing the risk of over suppression of plasma intact parathyroid hormone levels in a patient undergoing treatment for elevated levels of plasma intact parathyroid hormone, by administering 1α-hydroxyvitamin D₂ in an amount sufficient to decrease elevated intact parathyroid hormone levels while avoiding an abnormally low bone turnover rate. Treatment with certain vitamin D compounds, e.g. calcitriol, has resulted in over suppression of PTH levels. See e.g. Malluche et al., Nephrol. Dial. Transplant., 2004, 19 (suppl_{—}1), p. 9-13. incorporated herein by reference. Low levels of PTH gives rise to abnormally low bone turnover rate. Treatment in accordance with the present invention decreases the risk of over suppression and abnormally low bone turnover rate. In other words, treatment of a patient in need with 1α-hydroxyvitamin D₂ ameliorates elevated intact serum PTH with a reduced risk of over suppression of PTH and of abnormally low bone turnover rate.

As used herein the term “1,25 dihydroxyvitamin D” refers to the combined amount of 1,25 dihydroxyvitamin D₂ and 1,25-dihydroxyvitamin D₃.

As used herein the term “patient's normal historical physiological range of 1,25 dihydroxyvitamin D” refers to the average blood concentration range of 1,25 dihydroxyvitamin D of a patient taken from at least two annual or biannual readings of the patient's serum 1,25 dihydroxyvitamin D levels taken while the patient's kidneys are healthy and before the patient reaches the age of 40.

As used herein the term “hypercalcemia” refers to condition in a patient wherein the patient has corrected serum levels of calcium above 10.7 mg/dL. Normal corrected serum levels of calcium for a human are between about 8.6 to 10.7 mg/dL.

As used herein the term “hyperphosphatemia” refers to a condition in a patient having normal kidney function, or stage 1-4 CKD, wherein the patient has serum phosphorous levels above 5 mg/dL. In a patient who has stage 5 CKD, hyperphosphatemia occurs when the patient has serum levels above 5.5 mg/dL. Normal values for serum phosphorous in a human are 2.4-5 mg/dL.

As used herein the term “over suppression of plasma intact parathyroid hormone” refers to a condition in a patient having normal kidney function, or stage 1-3 CKD, wherein the patient has levels of plasma intact parathyroid hormone below 65 pg/mL. In a patient having stage 4 CKD, over suppression of plasma intact parathyroid hormone occurs when the patient has levels of plasma intact parathyroid hormone below 70 pg/mL. In a patient having stage 5 CKD, over suppression of plasma intact parathyroid hormone occurs when the patient has levels of plasma intact parathyroid hormone below 150 pg/mL.

As used herein the term “abnormally low bone turnover rate” refers to a condition in a patient wherein the rate of bone resorption is greater than the rate of bone formation. Bone turnover rate can be determined serum bone-specific markers such as alkaline phosphatase, N- and C-telopeptides, and osteocalcin. If most of these markers are shown in a patient to be below normal reference ranges, the patient has an abnormally low bone turnover rate. For bone-specific alkaline phosphatase: males above the age of 35 have normal levels of 15.0 u/L to 41.3 u/L, females 25-44 years have normal levels of 11.6 u/L to 29.6 u/L, and females over 45 years have normal levels of 14.2 u/L to 42.7 u/L. For serum osteocalcin: males have normal ranges of 11 ng/mL to 46 ng/mL, females (premenopausal) have normal ranges of 12 ng/mL to 41 ng/mL, and females (postmenopausal) have normal ranges of 20 ng/mL to 48 ng/mL. For N-Telopeptides: males have normal ranges of 5.4-24.2 nM BCE and females have normal ranges of 6.2-19.0 nM BCE.

It also is understood that any numerical value recited herein includes all values from the lower value to the upper value. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between the lowest value and the highest value enumerated are to be considered to be expressly stated in this application.

1α-hydroxyvitamin D₂ is also useful as active compounds in pharmaceutical compositions. The pharmacologically active analogs of this invention can be processed in accordance with conventional methods of pharmacy to produce pharmaceutical agents for administration to patients, e.g., in admixtures with conventional excipients such as pharmaceutically acceptable organic or inorganic carrier substances suitable for parenteral, enteral (e.g., oral), topical or transdermal application which do not deleteriously react with the active compounds. Suitable pharmaceutically acceptable carriers include, but are not limited to, water, salt (buffer) solutions, alcohols, gum arabic, mineral and vegetable oils, benzyl alcohols, polyethylene glycols, gelatin, carbohydrates such as lactose, amylose or starch, magnesium stearate, talc, silicic acid, viscous paraffin, perfume oil, fatty acid monoglycerides and diglycerides, pentaerythritol fatty acid esters, hydroxy methylcellulose, polyvinyl pyrrolidone, etc.

The pharmaceutical preparations can be sterilized and, if desired, mixed with auxiliary agents, e.g., lubricants, preservatives, stabilizers, wetting agents, emulsifiers, salts for influencing osmotic pressure, buffers, coloring, flavoring and/or aromatic active compounds. If a pharmaceutically acceptable solid carrier is used, the dosage form of the analogs may be tablets, capsules, powders, suppositories, or lozenges. If a liquid carrier is used, soft gelatin capsules, transdermal patches, aerosol sprays, topical creams, syrups or liquid suspensions, emulsions or solutions may be the dosage form.

For parenteral application, particularly suitable are injectable, sterile solutions, preferably oily or aqueous solutions, as well as suspensions, emulsions, or implants, including suppositories. Ampoules are convenient unit dosages.

For enteral application, particularly suitable are tablets, dragees, liquids, drops, suppositories, or capsules such as soft gelatin capsules. A syrup, elixir, or the like can be used wherein a sweetened vehicle is employed.

Sustained or directed release compositions can be formulated, e.g., liposomes or those wherein the active compound is protected with differentially degradable coatings, such as by microencapsulation, multiple coatings, etc. It is also possible to freeze-dry the new compounds and use the lypolizates obtained, for example, for the preparation of products for injection. Transdermal delivery of pharmaceutical compositions of the compounds of the invention is also possible.

For topical application, there are employed as nonsprayable forms, viscous to semi-solid or solid forms comprising a carrier compatible with topical application and having a dynamic viscosity preferably greater than water. Suitable formulations include, but are not limited to, solutions, suspensions, emulsions, creams, ointments, powders, liniments, salves, aerosols, etc., which are, if desired, sterilized or mixed with auxiliary agents, e.g., preservatives, etc.

It is possible, if desired, to produce the metabolites of certain ones of the compounds of the invention, in particular by nonchemical means. For this purpose, it is possible to convert them into a suitable form for administration together with at least one vehicle or auxiliary and, where appropriate, combined with one or more other active compounds.

The dosage forms may also contain adjuvants, such as preserving or stabilizing adjuvants. They may also contain other therapeutically valuable substances or may contain more than one of the compounds specified herein and in the claims in admixture.

As described hereinbefore, 1α-hydroxyvitamin D₂ is preferably administered to the human patients in oral dosage formulation. 1α-hydroxyvitamin D₂ is released from the oral dosage formulation, and is absorbed from the intestine into the blood.

The dosage of 1α-hydroxyvitamin D₂ in accordance with the present invention can be done on an episodic basis, suitably from daily, 1 to 3 times a week, or once every twelve weeks. Suitably the dosage of 1α-hydroxyvitamin D₂ is about 0.5 μg to about 200 μg per week, about 0.5 μg to about 50 μg per week, or about 1.5 μg to about 2.5 μg per week. Suitably 1α-hydroxyvitamin D₂ can be given in a unit dosage form between about 0.5 μg to about 100 μg, or about 0.5 μg to about 10 μg in a pharmaceutically acceptable carrier per unit dosage. Suitably unit dosages of 1α-hydroxyvitamin D₂ include 0.5 μg, 1.5 μg and 2.5 μg doses. Episodic doses can be a single dose or, optionally, divided into 2-4 subdoses which, if desired, can be given, e.g., twenty minutes to an hour apart until the total dose is given.

Those of ordinary skill in the art will readily optimize effective doses and co-administration regimens as determined by good medical practice and the clinical condition of the individual patient. Regardless of the manner of administration, it will be appreciated that the actual preferred amounts of active compound in a specific case will vary according to the efficacy of the specific compound employed, the particular compositions formulated, the mode of application, and the particular situs and organism being treated. For example, the specific dose for a particular patient depends on age, sex, body weight, general state of health, on diet, on the timing and mode of administration, on the rate of excretion, and on medicaments used in combination and the severity of the particular disorder to which the therapy is applied. Dosages for a given patient can be determined using conventional considerations, e.g., by customary comparison of the differential activities of the subject compounds and of a known agent, such as by means of an appropriate conventional pharmacological protocol. A physician of ordinary skill can readily determine and prescribe the effective amount of the drug required to counter or arrest the progress of the condition. Optimal precision in achieving concentrations of drug within the range that yields efficacy without toxicity requires a regimen based on the kinetics of the drug's availability to target sites. This involves a consideration of the distribution, equilibrium, and elimination of a drug. The dosage of active ingredient in the compositions of this invention may be varied; however, it is necessary that the amount of the active ingredient be such that an efficacious dosage is obtained. The active ingredient is administered to patients (animal and human) in need of treatment in dosages that will provide optimal pharmaceutical efficacy.

Bulk quantities of the vitamin D analogs in accordance with the present invention can be readily obtained in accordance with the many widely known processes, e.g., as described in U.S. Pat. Nos. 3,907,843; 4,195,027; 4,202,829; 4,234,495; 4,260,549; 4,555,364; 4,554,106; 4,670,190; and 5,488,120; WO 94/05630, and Strugnell et al., 310 Biochem. J. 233-241 (1995), all of which are herein fully incorporated by reference.

The present invention is further explained by the following examples which should not be construed by way of limiting the scope of the present invention.

The following examples demonstrate that 1α-(OH)D₂ are effective in reducing or preventing elevated blood PTH levels as well as maintaining therapeutic levels of serum calcium, serum phosphorous, and serum 1,25 dihydroxyvitamin D.

### EXAMPLE 1

### Use of 1α-hydroxyvitamin D₂ for Treating Human Osteoporosis

1α-(OH)D₂ was used to treat osteoporosis was confirmed in a study involving 60 postmenopausal osteoporotic outpatients. The selected subjects had ages between 60 and 70 years, and exhibited L2-L3 vertebral BMD between 0.7 and 1.05 g/cm², as determined by dual-energy x-ray absorptiometry (DEXA). Exclusion criteria encompassed significant medical disorders and recent use of medications known to affect bone or calcium metabolism.

On admission to the study, each subject was assigned at random to one of two treatment groups; one group received up to a 104-week course of therapy with 1α-(OH)D₂; the other received only placebo therapy. All subjects received instruction on selecting a daily diet containing 700-900 mg of calcium and were advised to adhere to this diet over the course of the study. Compliance to the diet was verified at regular intervals by 24-hour food records and by interviews with each subject.

During the treatment period, subjects from one group orally self-administered 1α-(OH)D₂ at an initial dosage of 1.0 μg/day for one week, and increased the dosage to 2.0, 3.0, 4.0 μg/day in each of the following weeks, to a maximum dosage of 5.0 μg/day. The dosage for any given subject was increased in this way until the rate of urinary calcium excretion was elevated to approximately 275-300 mg/24 hours, at which point the subject held the dosage constant at the highest level attained. Subjects from the second group self-administered a matching placebo medication every day, titrating the apparent dosage upwards in the same manner as subjects being treated with 1α-(OH)D₂.

Spinal and femoral neck BMD were measured in all subjects by DEXA at the beginning of the study, and at six-month intervals thereafter. Intestinal calcium absorption was estimated in all subjects by a single isotope technique at the beginning of the study, and at 12-month intervals. Serum levels of vitamin D metabolites were determined by radioreceptor binding assays at baseline and at six-month intervals. Serum osteocalcin, serum PTH and urine hydroxyproline also were determined at baseline and at six-month intervals.

Other blood and urine chemistries were monitored at regular intervals during the treatment period. These chemistries included serum calcium, serum ionized calcium, urine calcium, blood urea nitrogen, serum creatinine and creatinine clearance. Kidney-ureter-bladder (KUB) x-rays were obtained at baseline and at 12-month intervals thereafter.

Sixty subjects enrolled in what was originally intended to be a 52-week study. Of these 60 subjects, 55 completed one year of treatment (28 active; 27 placebo); and 41 subjects completed an optional second year of treatment.

The average prescribed dosage for subjects who received 1α-(OH)D₂ was 4.2 μg/day at 52 weeks and 3.6 μg/day at 104 weeks. The average prescribed dosage for placebo subjects was an apparent 4.8 μg/day at 52 weeks and 4.8 μg/day at 104 weeks.

One subject failed to comply with the prescribed dosage of test drug, as confirmed by an absence of serum 1α,25-(OH)₂D₂ at any time during the study. Data for this subject were excluded from analysis. Three patients were diagnosed with hyperparathyroidism when the PTH assays were completed (in batch) at the study's conclusion; data for these subjects were excluded from analysis. No subjects were excluded from analysis for noncompliance with the required dietary calcium intake of 700-900 mg/day.

Marked hypercalcemia (>10.8 mg/dL) occurred in one subject in association with an intercurrent illness. The prescribed dosage of 1α-(OH)D₂ at the time of this episode was 5.0 μg/day. Moderate hypercalcemia (10.4-10.8 mg/dL) occurred in two subjects over the course of the study at prescribed dosages of 5.0 μg/day. Mild hypercalcemia (10.2-10.4 mg/dL) occurred in four subjects in the first year and in two subjects in the second year. Hypercalciuria was observed occasionally over the two-year study in 17 subjects treated with 1α-(OH)D₂.

Mean serum calcium was approximately 0.1 to 0.2 mg/dL higher in subjects treated with 1α-(OH)D₂ than in subjects treated with placebo. Mean serum ionized calcium was approximately 0.05 to 0.10 mg/dL higher in subjects treated with 1α-(OH)D₂.

Mean urine calcium increased during the initial titration period in a dose-response fashion. After titration, mean urine calcium was 50 to 130% higher (P<001) with 1α-(OH)D₂ treatment than with placebo treatment.

Bone mineral density (BMD) in the L2-L4 vertebrae progressively increased with 1α-(OH)D₂ treatment and decreased with placebo treatment over the two-year study. The difference in spinal BMD between the treatment groups became statistically significant (P<0.05) after 24 months of treatment. Similar changes were observed in femoral neck BMD with statistically significant differences observed after 18 months (P<0.001) and 24 months (P<0.05) of treatment.

Intestinal absorption of orally administered Ca increased by 40% (P<0.001) after 52 weeks of 1α-(OH)D₂ therapy, and by 29% (P<0.5) after 104 weeks of 1α-(OH)D₂ therapy, relative to placebo control.

Treatment with 1α-(OH)D₂ caused progressive increases in mean serum total 1α,25-(OH)₂D₃ from 21% (P<0.05) at six months to 49% (P<0.01) at 24 months relative to placebo therapy. This increase resulted from a dramatic rise in serum 1α,25-(OH)₂D₂ which was partially offset by a 50+% decrease in serum 1α,25-(OH)₂D₃.

Serum levels of PTH decreased with 1α-(OH)D₂ therapy by 17% at 52 weeks and by 25% at 1-4 weeks, relative to placebo therapy.

### EXAMPLE 2

### 1α-hydroxyvitamin D₂ for Treating Subjects with Chronic Kidney Disease with Elevated Blood PTH

1α-(OH)D₂ (doxercalciferol) was used as a treatment for hyperparathyroidism associated with chronic kidney disease was confirmed in a study involving 55 adults, ages 18-85 years, with mild to moderate chronic kidney disease. The subjects had plasma intact parathyroid hormone (iPTH) levels above 85 pg/mL and completed an eight-week baseline period and then 24 weeks of therapy with either orally administered doxercalciferol or placebo.

The initial dose of test drug was 2 capsules daily (totaling 1.0 μg for subjects randomized to doxercalciferol treatment), with increases in steps of one capsule per day permitted after four weeks. The maximum dosage was limited to 10 capsules per day (5.0 μg/day of doxercalciferol). Subjects were monitored at regular intervals for plasma iPTH, serum calcium and phosphorus, 24-hour and fasting urinary calcium, bone-specific serum markers, plasma total 1α,25-(OH)₂D, and routine blood chemistries and hematologies. Glomerular filtration rate (GFR) was measured prior to beginning the treatment and at study termination. No physical or biochemical differences were detectable between the two treatment groups prior to starting treatment.

During doxercalciferol treatment, mean serum 1,25-dihydroxyvitamin D rose nearly 50% from baseline levels whereas no change in was observed in the placebo group. Mean plasma iPTH progressively decreased from baseline levels, reaching maximum suppression of 45.6% after 24 weeks (p<0.001). No corresponding changes in mean iPTH were observed during placebo treatment. Mean iPTH was lower in subject receiving doxercalciferol versus placebo at all treatment weeks (p<0.001). Serum C- and N-telopeptides and bone-specific alkaline phosphate decreased with doxercalciferol treatment relative to baseline and placebo treatment (p<0.01). No differences between treatment groups were observed with regard to renal function and rates of adverse events.

Pre-dialysis patients exhibiting secondary hyperparathyroidism associated with mild to moderate chronic kidney disease were recruited to participate in two multicenter, double-blinded, placebo-controlled studies conducted according to a common protocol. On enrollment, each subject was assigned, at random, in double-blinded fashion, to one of two treatment groups. Both treatment groups completed an 8-week Baseline Period (Weeks −8 to 0) and then underwent therapy with either orally administered doxercalciferol or placebo for a 24-week Treatment Period (Weeks 1 to 24). Irrespective of treatment group assignment, each subject discontinued any 1α,25-dihydroxivitamin D₃ (1α,25(OH)₂D₃) therapy for the duration of the study. Throughout the Baseline Period and the subsequent Treatment Period, subjects were monitored at regular intervals for plasma iPTH, serum calcium, serum phosphorus, and 24-hour and fasting urinary calcium, phosphorus and creatinine. Routine blood chemistries and hematologies, bone-specific serum markers, and plasma total 1α,25(OH)₂D were also monitored at selected intervals. Glomerular filtration rate (GFR) was measured prior to beginning treatment and at termination.

Subjects qualified for inclusion in the Baseline Period if they were aged 18 to 85 years, had mild to moderate CR1 with serum creatinine between 1.8 to 5.0 mgldL (for men) or 1.6 to 4.0 mg/dL (for women), and had elevated plasma iPTH values (>85 pg/mL). Subjects receiving ongoing treatment with estrogen were required to maintain the same estrogen dosing regimen throughout the study. Subjects who began dialysis treatment or underwent renal transplantation were required to prematurely terminate participation. Screened patients were excluded if they had a current history of alcohol or drug abuse, were pregnant, possibly pregnant, or nursing, had a history of idiopathic urinary calcium stone disease, had undergone renal transplant surgery, or had received treatment in the past year with anticonvulsants, oral steroids, bisphosphonates, fluoride, or lithium. Patients were also excluded who had hypercalcemia, hyperthyroidism, sarcoidosis, malignancy requiring chemotherapy, hormonal therapy and/or radiation treatment, chronic gastrointestinal disease (i.e., malabsorption, surgery affecting absorption, and chronic ulcerative colitis), hepatic impairment, or any other condition which may have put the patient at undue risk. Qualified, enrolled subjects were precluded from entering the Treatment Period and prematurely terminated participation if they exhibited during the Baseline Period a urinary protein ≧4 grams/24 hours associated with a serum albumin ≦3.5 grams/dL, a urine calcium level (at Week −4) above 150 mg/24 hours, or a markedly elevated serum creatinine value (>5.0 mg/dL for men or: >4.0 mg/dL for women).

The two studies were conducted under double-blind conditions in each geographical region. Assignments of subjects to the two treatment groups were made randomly, by geographical region, in order of enrollment. The randomization was accomplished in subgroups of size 10, 5 subjects assigned to each of the two treatment groups. The randomization was performed by an independent statistician using the Statistical Analysis System (SAS).

1α-hydroxyvitamin D₂ (available as doxercalciferol from Bone Care International) was formulated for oral administration as soft elastic gelatin capsules in units of 0.5 mcg/capsule. Matching placebo capsules contained no doxercalciferol and were formulated from the same inactive ingredients in identical proportions. The inactive ingredients, in order of decreasing weight, were as follows: fractionated coconut oil, gelatin, glycerin, titaninum dioxide, FD&C Red #40, D&C Yellow #10, ethanol and butylated hydroxyanisole (BHA).

The initial dose of test drug (doxercalciferol or placebo) was 2 capsules (totaling a 1.0 μg dose for subjects receiving doxercalciferol) every day before breakfast. This dosage was increased as necessary at monthly intervals, to suppress plasma iPTH levels by at least 30% from baseline. Dosage increases in steps of one capsule (0.5 μg) per day were permitted only if serum calcium was ≦9.6 mg/dL, serum phosphorus was ≦5.0 mg/dL, urine calcium was ≦200 mg/24 hours, and fasting urine calcium/urine creatinine ratio (urine Ca/Cr) was ≦0.25. The maximum dosage was limited to 10 capsules/day (5.0 μg/day of doxercalciferol or 35.0 μg/week).

Subjects suspended treatment if they developed moderate hypercalcemia (serum calcium >10.7 mg/dL corrected for serum albumin) and/or hypercalciuria (urine calcium>200 mg/24 hours or fasting urine Ca/Cr>0.25) during the Treatment Period. Such subjects were monitored weekly until the serum or urine calcium was normalized (<10.2 mg/dL and/or<150 mg/24 hours or<0.25, respectively) and then resumed test drug dosing at a reduced rate with adjustment in their consumption of calcium-based phosphate binder, as appropriate. Subjects who developed mild hypercalcemia (serum calcium of 10.3 to 10.7 mg/dL) or hyperphosphatemia (serum phosphorus>5.0 mg/dL) during the Treatment Period adjusted their consumption of calcium-based phosphate binder and/or reduced their test drug dosage. At the discretion of the site Investigator(s), the dosage of calcium-based phosphate binder was increased for subjects who presented with hypocalcemia (≧9.0 mg/dL).

If one of the dosage levels was not optimum for a given subject (i.e., maintaining plasma iPTH suppression ≧30% from baseline and >15 pg/mL), the site Investigator(s) could vary the daily dosage administered according to a defined schedule (e.g., alternating dose of 1.0 μg with 0.5 μg) so that the total weekly dosage was optimized to the subject's needs.

Blood samples for analysis of serum chemistries, hematology and plasma iPTH were taken. Plasma iPTH samples were analyzed using a two-site immunoradiometric assay (IRMA).

The 24-hour urine samples for total protein and the 24-hour and spot urine samples for calcium, phosphorus, and creatinine were processed at the clinical sites. Urine samples for calcium, phosphorus and creatinine were acidified to a pH<2.0 using 6M HCL. Duplicate 4-mL aliquots of each urine sample were analyzed.

Blood samples for serum osteocalcin, bone-specific alkaline phosphatase, serum C-telopeptide (sCTx) and serum N-telopeptide (sNTx) were collected at the clinical sites. Triplicate 1-mL aliquots of serum from each sample were analyzed. All samples obtained from each subject for a given parameter were analyzed together in the same batch.

Blood samples for serum total 1α,25-dihydroxyvitamin D were analyzed. Serum samples from each subject were analyzed batchwise by means of radioreceptor assay following high-performance liquid chromatography.

GFR was determined at baseline and at termination by the Technetium or lothalmate (Glofill®) method. Each site used the same standardized method among all subjects at that study site. Serial blood and urine samples collected for GFR determination were analyzed on site or were sent on ice to the Cleveland Clinic in Cleveland, OH for analysis.

Baseline values for all parameters were defined as the mean of the data collected during Weeks −4 and 0 of the Baseline Period. A positive response was defined as a reduction in mean plasma iPTH at Weeks 20 and 24 of ≧30% from baseline. At each time point, descriptive statistics were calculated, including n, mean, standard deviation, and standard error.

Also, the significance of the mean difference from baseline at each time point was assessed by paired t-test. This assessment was performed separately for each treatment group, with missing values being replaced by the last observation carried forward (LOCF).

The treatment groups were compared at baseline and at each subsequent time point, and the significance of differences in means was assessed via two-sample t-test. For certain parameters, the data were recalculated as a percent of baseline and the analyses performed on these percentages instead of on the absolute data values.

All of the above analyses were performed on an intent-to-treat basis only, meaning that all subjects who received test drug were evaluated for statistical purposes. The protocol allowed for a per-protocol analysis that excluded subjects with low dosing compliance (<80% of prescribed doses). However, this analysis was not completed since compliance to the prescribed dosages was ≧80%, with few exceptions.

Seventy-two subjects were enrolled into the Baseline Period. Of the 72 enrolled subjects, 55 (76%) were admitted into the Treatment Period of the study. Seventeen subjects (24%) terminated or were disqualified during the Baseline Period and were precluded from entering the Treatment Period. Of these, eight subjects exhibited urine total protein levels ≧4 grams/24 hours associated with a serum albumin ≦3.5 grams/dL, three subjects had a markedly elevated serum creatinine (>5.0 mg/dL for men or>4.0 mg/dL for women) at either of the first two washout visits (Weeks −8 or −4), one subject demonstrated a serum creatinine level lower than that allowed by the inclusion criterion, three subjects declined to continue participating for personal reasons, and two experienced SAEs and were discontinued prematurely.

Nine subjects discontinued after entering and before completing the Treatment Period. One of the subjects relocated out of the area where the study was being conducted, one was found to have an intestinal malabsorption disorder, six experienced SAEs leading to discontinuation, and one experienced a non-serious adverse event leading to discontinuation.

The 55 subjects enrolled into the Treatment Period had physical and biochemical characteristics within the specified acceptable ranges and were otherwise qualified to participate in the study. These subjects had ages between 36 and 84 years (mean±SE=64.6±8.7 years). Forty-five subjects were men and 10 were women; 22 were African-Americans, 28 were Caucasians, four were Hispanics, and one was self-designated as “Other”. A comparison of the subjects assigned to active and placebo treatment with regard to physical and biochemical characteristics at baseline is provided in Table I. There were no statistically significant differences between these two groups for the tabulated characteristics.

Dosing compliance ‘was above 80% in 52 of the 55 treated subjects. Dosing compliance was 71% in one subject randomized to placebo treatment and 79% in another subject randomized to active treatment. A. third subject (active group) achieved only a 67% dosing compliance due to an adverse event unrelated to the drug. This subject discontinued participation in the study at Week 5.

The average (±SE) weekly prescribed dosages of test medication remained at the initial level of 2.0 capsules per day (1.0 mcg for subjects receiving doxercalciferol) for the first month, as required by the study protocol. Thereafter, the mean dose in the active group increased, reaching 3.28±0.39 capsules per day (1.61±0.20 mcg/day) by Week 24 (range: 1.0 to 3.5 meg/day). The mean dose in the placebo group also increased, reaching 5.13±0.49 capsules per day by Week 24 (range: 2.0 to 10.0 capsules/day). The mean weekly prescribed dose trended higher in the placebo group from Week 6 through Week 24, with the difference reaching statistical significance at Weeks 20 and 24.

Decreases in test drug dosage occurred in some subjects. The primary reason for a decrease in prescribed dose was suppression of plasma iPTH by more than 30% from baseline level. In a few cases, dosing with test medication was suspended for intercurrent illness and restarted, when possible, at the same level.

At baseline, mean (±SE) plasma PTH was 219.1±22.3 pg/mL in the active group, with a range from 57 to 583 pg/mL and 171±14 pg/mL in the placebo group, with a range from 63 to 330 pg/mL. There was no difference in baseline iPTH levels between treatment groups (p=0.07). With initiation of doxercalciferol treatment, mean iPTH decreased to 165±15 pg/mL at Week 4 (p=O.O01 vs. baseline) and continued to decrease through Week 24, at which time the mean iPTH was 118±17 pg/mL (p<0.001 vs. baseline). In contrast, mean iPTH remained unchanged from baseline levels in the placebo group throughout the entire Treatment Period (p≧0.17), ending at 167±15 at Week 24. Mean iPTH was significantly lower in subjects receiving doxercalciferol at Weeks 16-24 (p<0.05 vs. placebo).

At the end of treatment, 20 (74%) of 27 subjects in the active group had achieved plasma iPTH suppression of ≧30% from baseline. This positive end-point response was based on the mean of plasma iPTH determinations at Weeks 20 and 24. Three of the other seven subjects had iPTH reductions of 24.0%, 24.2%, and 19.6%, respectively, and one subject had an increase in iPTH of 3.9%. The remaining three subjects showed the following responses: one discontinued participation in Week 17, at which time plasma iPTH was suppressed by 44.4%; another discontinued doxercalciferol treatment in Week 8, at which time plasma iPTH was suppressed by 27.9% from baseline; the third subject discontinued treatment in Week 5, at which time iPTH was increased by 22.8%. Only two (7.1%) of the 28 subjects treated with placebo achieved iPTH suppression of ≧30%.

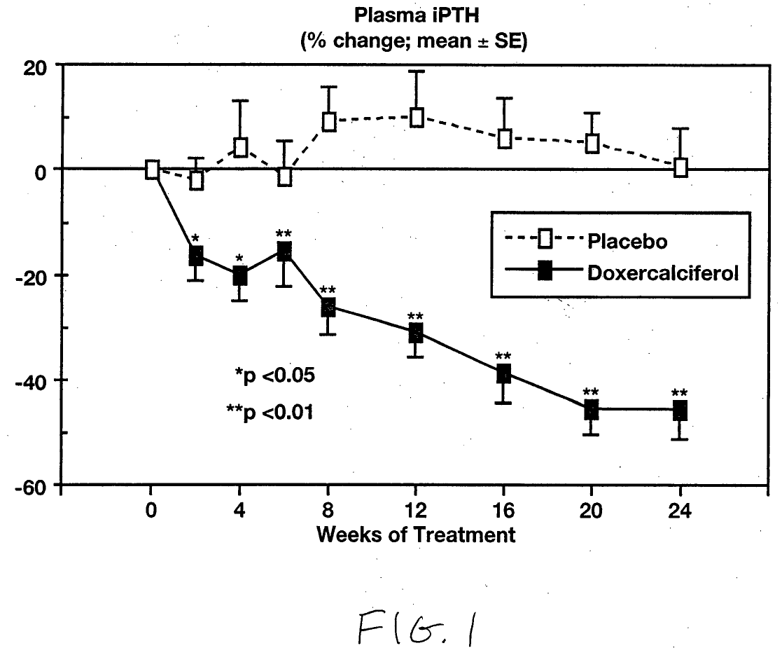

Subjects randomized to doxercalciferol treatment exhibited progressively greater reductions in mean plasma iPTH during the course of the Treatment Period (see FIG. 1) Mean reduction of iPTH was 26.3% from baseline at Week 8, and 45.6% at Week 24. Mean iPTH reductions were significant (p<0.05 vs. baseline) from Week 2 through Week 24. Subjects randomized to placebo treatment exhibited no changes in mean plasma iPTH expressed as a percentage of baseline (p>0.17). Mean iPTH reduction was significantly greater in the active group at all Weeks except Week 6 (p<0.05).

Baseline mean (±SE) serum calcium level was 8.74±0.12 mg/dL in the active group and 8.82±0.13 mg/dL in the placebo group (NS). At Week 24, mean serum calcium was 9.14±0.11 mg/dL in the active group and 8.95±0.13 mg/dL in the placebo group (NS). The increase in mean serum calcium from baseline was significant (p<0.05) at Week 4 and at Weeks 12-24 in subjects treated with doxercalciferol, but not in subjects treated with placebo. Mean serum calcium differed between the treatment groups only at Week 20 (p<0.04).

At baseline, mean (±SE) serum phosphorus level was 4.02±0.15 mg/dL in the active group and 3.89±0.13 mg/dL in the placebo group (pNS). At Week 24, mean serum phosphorus was 4.27±0.13 mg/dL in the active group and 3.92±0.12 mg/dL in the placebo group (p=NS).

Two episodes of hypercalcemia (determined as corrected serum calcium>10.7 mg/dL) occurred in one subject receiving doxercalciferol treatment, with onsets in Week 4 and Week 16, respectively. The maximum serum calcium recorded during each of these episodes was 10.9 and 11.0 mg/dL, respectively, and the duration of each episode was 5 and 8 weeks, respectively. This subject had a serum calcium of 10.4 mg/dL at baseline and had exhibited serum calcium as high as 10.7 mg/dL during the Baseline Period. One episode of hypercalcemia (defined as corrected serum calcium>10.7 mg/dL) occurred in one subject receiving placebo treatment with onset in Week 12. The maximum serum calcium recorded during this episode was 10.9 mg/dL, and the duration of the episode was approximately 8 weeks. There were 9 episodes of hyperphosphatemia (defined as serum phosphorus>5.0 mg/dL) in 9 subjects during the Baseline Period. During the Treatment Period, there were 15 episodes of hyperphosphatemia in 10 subjects receiving active treatment and 9 episodes in 8 subjects receiving placebo treatment. Only one episode of Ca×P>65 occurred during the Treatment Period in one subject receiving placebo treatment.

No episodes of hypercalciuria (defined as 24-hour urine calcium excretion greater than 200 mg or fasting urine Ca/Cr ratio above 0.25) occurred during the Treatment Period in either the active or placebo groups.

Mean alkaline phosphatase was reduced significantly from baseline in the active group at Weeks 16 and 24 (p<0.05), but was not lowered in the placebo group during the Treatment Period.

Subjects treated with doxercalciferol showed mean reductions in serum bone-specific alkaline phosphatase (BSAP) from baseline of 19.7±3.7% by Week 16 (p<O.OI) and 27.9±4.6% by Week 24 (p<O.OI). Subjects treated with placebo showed no change in BSAP relative to baseline at any treatment week. Mean BSAP reductions differed significantly between treatment groups from Weeks 8 to 24 (p≦O.OI). Similar reductions were observed in serum N- and C-telopeptides with doxercalciferol treatment. Mean serum osteocalcin trended upward from baseline with doxercalciferol treatment by nearly 10% at Week 4 and then progressively declined from baseline by about 20% at Week 24. Mean serum total 1,25-dihydroxyvitamin D levels increased significantly from baseline in the active group at all treatment weeks but did not differ significantly between groups at any treatment week.

All subjects treated with 1α-(OH)D₂ remained non-uremic and maintained controlled urine calcium (range of 4.0 to 176.0 mg/24 hr). Serum calcium for these subjects remained in the range of 7.6 to 10.5 mg/dL, with the exception of one subject who had pre-treatment serum calcium determinations as high as 10.7 mg/dL, which increased to 11.0 mg/dL during treatment. This patient had no elevation of urine calcium and no abnormally low serum bone-specific markers (i.e., serum bone-specific alkaline phosphatase, N- and C-telopeptides, and osteocalcin).

### EXAMPLE 3

### 1α-hydroxyvitamin D₂ for Treating Subjects with Low Serum Calcium

1α-(OH)D₂ (doxercalciferol) is used as a treatment for subjects with low serum calcium in a study involving 50 adults, ages 18-85 years.

The subjects have serum calcium levels below 8.6 mg/dL and complete an eight-week baseline period and then 24 weeks of therapy with orally administered doxercalciferol.

The initial dose of 1α-(OH)D₂ is 2 capsules daily (totaling 1.0 μg), with increases in steps of one capsule per day permitted after four weeks. The maximum dosage is limited to 10 capsules per day (5.0 μg/day of doxercalciferol). Subjects are monitored at regular intervals for plasma iPTH, serum calcium and phosphorus, 24-hour and fasting urinary calcium, bone-specific serum markers, plasma total 1α,25-(OH)₂D, and routine blood chemistries and hematologies.

After the 24 week treatment period the subjects treated with 1α-(OH)D₂ show average serum phosphorous levels between about 2.4-5 mg/dL, average corrected serum calcium levels between about 8.6 to 10.7 mg/dL, average intact serum parathyroid hormone levels between about 65 pg/mL and 110 pg/mL, and average blood concentrations of 1,25-dihydroxyvitamin D between about 20 pg/mL to 60 pg/mL. Testing of serum 1,25-dihydroxyvitamin D levels between doses of 1α-(OH)D2 shows that serum 1,25-dihydroxyvitamin D levels in the patients are within the patients normal historical physiological range for 1,25-dihydroxyvitamin D. Levels of serum bone-specific markers alkaline phosphatase, N- and C-telopeptides, and osteocalcin in patients show average normal levels of these markers.

### EXAMPLE 4

### 1α-hydroxyvitamin D₂ for Treating Subjects with Low Serum 1,25-Dihydroxyvitamin D₂

1α-(OH)D₂ (doxercalciferol) is used as a treatment for subjects with low serum 1,25-dihydroxyvitamin D in a study involving 50 adults, ages 18-85 years.

The subjects have serum 1,25-dihydroxyvitamin D levels below 20 pg/dL and complete an eight-week baseline period and then 24 weeks of therapy with orally administered doxercalciferol.

The initial dose of 1α-(OH)D₂ is 2 capsules daily (totaling 1.0 μg), with increases in steps of one capsule per day permitted after four weeks. The maximum dosage is limited to 10 capsules per day (5.0 μg/day of doxercalciferol). Subjects are monitored at regular intervals for plasma iPTH, serum calcium and phosphorus, 24-hour and fasting urinary calcium, bone-specific serum markers, plasma total 1α,25-(OH)₂D, and routine blood chemistries and hematologies.

After the 24 week treatment period the subjects treated with 1α-(OH)D₂ show average serum phosphorous levels between about 2.4-5 mg/dL, average corrected serum calcium levels between about 8.6 to 10.7 mg/dL, average intact serum parathyroid hormone levels between about 65 pg/mL and 110 pg/mL, and average blood concentrations of 1,25-dihydroxyvitamin D between about 20 pg/mL to 60 pg/mL. Testing of serum 1,25-dihydroxyvitamin D levels between doses of 1α-(OH)D2 shows that serum 1,25-dihydroxyvitamin D levels in the patients are within the patients normal historical physiological range for 1,25-dihydroxyvitamin D. Levels of serum bone-specific markers alkaline phosphatase, N- and C-telopeptides, and osteocalcin in patients show average normal levels of these markers.

In summary, the present invention provides therapeutic methods for treating conditions associated with low blood concentrations of 1,25-dihydroxyvitamin D, elevated concentrations of PTH, elevated concentrations of serum phosphorous, and low concentrations of serum calcium. The methods are suitable for lowering elevated blood parathyroid hormone levels, or maintaining lowered blood PTH levels in subjects while maintaining normalized or targeted levels of serum calcium, serum phosphorous, and serum 1,25 dihydroxyvitamin D₂. The method of the present invention also includes reducing the risk of over suppression of PTH by administering to a subject in need thereof an amount of 1α-hydroxyvitamin D₂ to lower or maintain PTH levels while avoiding or preventing low bone turnover rate, i.e. adynamic bone disease.

While the present invention has now been described and exemplified with some specificity, those skilled in the art will appreciate the various modifications, including variations, additions, and omissions that may be made in what has been described. Accordingly, it is intended that these modifications also be encompassed by the present invention and that the scope of the present invention be limited solely by the broadest interpretation that lawfully can be accorded the appended claims.

All patents, publications and references cited herein are hereby fully incorporated by reference. In case of conflict between the present disclosure and incorporated patents, publications and references, the present disclosure should control.

1. A method of maintaining blood concentrations of 1,25-dihydroxyvitamin D in a patient at levels within the patient's normal historical physiological range without causing substantially increased risk of hypercalcemia, hyperphosphatemia or over suppression of plasma intact parathyroid hormone comprising administering to the patient an amount of 1α-hydroxyvitamin D₂ in episodic doses, wherein the levels of 1,25-dihydroxyvitamin D are maintained in the patient's normal historical physiological range between doses of 1α-hydroxyvitamin D₂.

2. The method of claim 1 wherein the patient suffers from chronic kidney disease.

3. A method in accordance with claim 2, wherein the chronic kidney disease is stage 1, stage 2, stage 3, or stage 4.

4. A method in accordance with claim 1 wherein the amount of the 1α-hydroxyvitamin D₂ is administered parenterally or orally in combination with a pharmaceutically acceptable carrier.

5. A method in accordance with claim 4 wherein the amount of 1α-hydroxyvitamin D₂ is administered parenterally.

6. A method in accordance with claim 5 wherein the amount of 1α-hydroxyvitamin D₂ is administered intravenously.

7. A method in accordance with claim 4 wherein the amount of 1α-hydroxyvitamin D₂ is administered orally.

8. A method in accordance with claim 4 wherein the 1α-hydroxyvitamin D₂ is co-administered with a phosphate binder.

9. A method in accordance with claim 5 wherein the 1α-hydroxyvitamin D₂ is administered is by intravenous injection, nasopharyngeally, or transdermally.

10. A method in accordance with claim 1 wherein the 1α-hydroxyvitamin D₂ is administered in a weekly dosage of about 0.5 μg to about 200 μg.

11. A method in accordance with claim 1 wherein the 1α-hydroxyvitamin D₂ is administered in a weekly dosage of about 0.5 μg to about 50 μg.

12. A method in accordance with claim 11, wherein the 1α-hydroxyvitamin D₂ is in a 0.5 μg per unit dosage form.

13. A method in accordance with claim 1 wherein the 1α-hydroxyvitamin D₂ is administered in a weekly dosage of about 1.5 to 2.5 μg.

14. A method in accordance with claim 13, wherein the 1α-hydroxyvitamin D₂ is in a 1.5 μg per unit dosage form.

15. A method in accordance with claim 13, wherein the 1α-hydroxyvitamin D₂ is in a 2.5 μg per unit dosage form.

16. A method of increasing blood concentrations of 1,25-dihydroxyvitamin D in a patient who has 1,25-dihydroxyvitamin D levels below the patient's normal historical physiological range without causing substantially increased risk of hypercalcemia, hyperphosphatemia or over suppression of plasma intact parathyroid hormone comprising administering to the patient an amount of 1α-hydroxyvitamin D₂ in episodic doses, wherein the levels of 1,25-dihydroxyvitamin D are maintained in the patient's normal historical physiological range between doses of 1α-hydroxyvitamin D₂.

17. The method of claim 17 wherein the patient suffers from chronic kidney disease.

18. A method in accordance with claim 17, wherein the chronic kidney disease is stage 1, stage 2, stage 3, or stage 4.

19. A method in accordance with claim 16 wherein the amount of the 1α-hydroxyvitamin D₂ is administered parenterally or orally in combination with a pharmaceutically acceptable carrier.

20. A method in accordance with claim 19 wherein the amount of 1α-hydroxyvitamin D₂ is administered parenterally.

21. A method in accordance with claim 20 wherein the amount of 1α-hydroxyvitamin D₂ is administered intravenously.

22. A method in accordance with claim 19 wherein the amount of 1α-hydroxyvitamin D₂ is administered orally.

23. A method in accordance with claim 19 wherein the 1α-hydroxyvitamin D₂ is co-administered with a phosphate binder.

24. A method in accordance with claim 20 wherein the 1α-hydroxyvitamin D₂ is administered is by intravenous injection, nasopharyngeally, or transdermally.

25. A method in accordance with claim 16 wherein the 1α-hydroxyvitamin D₂ is administered in a weekly dosage of about 0.5 μg to about 200 μg.

26. A method in accordance with claim 16 wherein the 1α-hydroxyvitamin D₂ is administered in a weekly dosage of about 0.5 μg to about 50 μg.

27. A method in accordance with claim 26, wherein the 1α-hydroxyvitamin D₂ is in a 0.5 μg per unit dosage form.

28. A method in accordance with claim 16 wherein the 1α-hydroxyvitamin D₂ is administered in a weekly dosage of about 1.5 to 2.5 μg.

29. A method in accordance with claim 28, wherein the 1α-hydroxyvitamin D₂ is in a 1.5 μg per unit dosage form.

30. A method in accordance with claim 28, wherein the 1α-hydroxyvitamin D₂ is in a 2.5 μg per unit dosage form.

31. A method of concurrently lowering or maintaining plasma intact parathyroid hormone levels, increasing or maintaining serum calcium levels, maintaining serum phosphorous levels, or increasing or maintaining serum 1,25-dihydroxyvitamin D levels in a human patient comprising administering to the patient an amount of 1α-hydroxyvitamin D₂.

32. The method of claim 31 wherein the patient has serum levels of plasma intact parathyroid hormone above 65 pg/mL before a first administration of 1α-hydroxyvitamin D₂.

33. The method of claim 31 wherein the patient has levels of serum calcium below 8.6 mg/dL before a first administration of 1α-hydroxyvitamin D₂.

34. The method of claim 31 wherein the patient levels of serum phosphorous are over 5 mg/dL before a first administration of 1α-hydroxyvitamin D₂.

35. The method of claim 31 wherein the patient has serum levels of 1,25-dihydroxyvitamin D below their normal historical physiological range before a first administration of 1α-hydroxyvitamin D₂.

36. A method in accordance with claim 31 wherein the amount of the 1α-hydroxyvitamin D₂ is administered parenterally or orally in combination with a pharmaceutically acceptable carrier.

37. A method in accordance with claim 36 wherein the amount of 1α-hydroxyvitamin D₂ is administered parenterally.

38. A method in accordance with claim 37 wherein the amount of 1α-hydroxyvitamin D₂ is administered intravenously.

39. A method in accordance with claim 36 wherein the amount of 1α-hydroxyvitamin D₂ is administered orally.

40. A method in accordance with claim 36 wherein the 1α-hydroxyvitamin D₂ is co-administered with a phosphate binder.

41. A method in accordance with claim 37 wherein the 1α-hydroxyvitamin D₂ is administered is by intravenous injection, nasopharyngeally, or transdermally.

42. A method in accordance with claim 31 wherein the 1α-hydroxyvitamin D₂ is administered in a weekly dosage of about 0.5 μg to about 200 μg.

43. A method in accordance with claim 31 wherein the 1α-hydroxyvitamin D₂ is administered in a weekly dosage of about 0.5 μg to about 50 μg.

44. A method in accordance with claim 43, wherein the 1α-hydroxyvitamin D₂ is in a 0.5 μg per unit dosage form.

45. A method in accordance with claim 31 wherein the 1α-hydroxyvitamin D₂ is administered in a weekly dosage of about 1.5 to 2.5 μg.

46. A method in accordance with claim 45, wherein the 1α-hydroxyvitamin D₂ is in a 1.5 μg per unit dosage form.

47. A method in accordance with claim 45, wherein the 1α-hydroxyvitamin D₂ is in a 2.5 μg per unit dosage form.

48. A method of reducing the risk of over suppression of plasma intact parathyroid hormone levels in a patient undergoing treatment for elevated levels of plasma intact parathyroid hormone, comprising administering 1α-hydroxyvitamin D₂ in an amount sufficient to decrease elevated intact parathyroid hormone levels while avoiding an abnormally low bone turnover rate.

49. A method in accordance with claim 48, wherein the chronic kidney disease is stage 1, stage 2, stage 3, stage 4 or stage 5.

50. A method in accordance with claim 48 wherein the amount of the 1α-hydroxyvitamin D₂ is administered parenterally or orally in combination with a pharmaceutically acceptable carrier.

51. A method in accordance with claim 50 wherein the amount of 1α-hydroxyvitamin D₂ is administered parenterally.

52. A method in accordance with claim 51 wherein the amount of 1α-hydroxyvitamin D₂ is administered intravenously.

53. A method in accordance with claim 50 wherein the amount of 1α-hydroxyvitamin D₂ is administered orally.

54. A method in accordance with claim 50 wherein the 1α-hydroxyvitamin D₂ is co-administered with a phosphate binder.

55. A method in accordance with claim 51 wherein the 1α-hydroxyvitamin D₂ is administered is by intravenous injection, nasopharyngeally, or transdermally.

56. A method in accordance with claim 48 wherein the 1α-hydroxyvitamin D₂ is administered in a weekly dosage of about 0.5 μg to about 200 μg.

57. A method in accordance with claim 48 wherein the 1α-hydroxyvitamin D₂ is administered in a weekly dosage of about 0.5 μg to about 50 μg.

58. A method in accordance with claim 57, wherein the 1α-hydroxyvitamin D₂ is in a 0.5 μg per unit dosage form.

59. A method in accordance with claim 48 wherein the 1α-hydroxyvitamin D₂ is administered in a weekly dosage of about 1.5 to 2.5 μg.

60. A method in accordance with claim 59, wherein the 1α-hydroxyvitamin D₂ is in a 1.5 μg per unit dosage form.

61. A method in accordance with claim 59, wherein the 1α-hydroxyvitamin D₂ is in a 2.5 μg per unit dosage form.
